(19) United States
(12) Patent Application Publication
(10) Pub. No.: US 2011/0054576 A1
(43) Pub. Date: Mar. 3, 2011

(54) Combined Portable Thermal and Vibratory Treatment Device

(76) Inventors: Ronni L. Robinson, Ambler, PA (US); Harry S. Sowden, Glenside, PA (US); Leo B. Kriksunov, Glenside, PA (US)

(21) Appl. No.: 12/553,164

(22) Filed: Sep. 3, 2009

(51) Int. Cl. A61F 7/00

(52) U.S. Cl. 607/108; 601/46

(57) ABSTRACT

The present invention is directed to a reusable pain relieving treatment device, such as a belt, and a disposable thermal insert comprising one or more thermally conductive component that extend from an inner surface of the device and are capable of transferring heat, cold and vibrations from disposable or reusable inserts.

### BACKGROUND OF THE INVENTION

The application of heat to the human body can decrease the viscosity of body fluids, loosen stiff muscles, improve blood flow to an affected area, facilitate tissue repair, and create a feeling of relaxation for many suffers of general joint and muscle pain. For some acute injuries, the immediate application of cold can numb pain, constrict blood vessels and mitigate an inflammatory response. The application of heat to the skin as a means to penetrate deeper into tissues has historically been used for pain relief of muscles and joints, as well as for the treatment of certain inflammatory conditions. The application of cold materials to the skin has also been used for similar treatments, especially for treating inflammatory responses such as joint inflammation. The application of vibratory materials to the skin has also been used for similar treatments.

Traditional heating devices have, in many instances, generated heat using chemical formulations, such as iron powder formulations, that oxidize when exposed to air. Commercially available thermal chemical formulation products are mainly categorized with disposable heat patches, which are available in loosely formed fabric that is filled with an exothermic composition. An alternate means of producing heat is by way of electrical heating elements that are attached to a power source. Since the desired time of treatment is often longer than 4 hours, in the case of an electrical source, the power source typically used in these types of devices is either an electrical wall outlet or a battery.

Other chemical heating devices include those products that incorporate heating portions into fabrics that can conform or are shaped to fit various parts of the body, such as the knee or the back, as shown in U.S. Pat. No. 6,074,413. In these cases, typically the entire product, including the garment and the heat providing exothermic formulation materials, is a disposable, unitary product. The chemical heating portion is not removable from such a unitary product, and therefore, the entire device is designed to be disposed of following use. Each use can typically last for between 6 to 12 hours, and a user can use 2 or 3 of these products over a 24-hour period. These types of products have the disadvantage of having loose powder formulations that do not always adequately conform to parts of the skin and do not conduct heat thoroughly to the skin since a woven or non-woven fabric surface is in contact with the skin.

Other types of devices, such as those shown in U.S. Pat. No. 5,484,366, exemplify elements that are not disposable, such as using a back belt with gel insert containers. In such a device the gel-inserts must be manually re-heated or cooled, taking more active participation by the user in order to be reusable. Similarly, the device shown in U.S. Pat. No. 6,416,534 uses a back belt with a flexible fabric, and a gel insert that is reheated using electrical heat. This type of device also involves active participation on the part of the user and a potential lag time in order to heat the gel-insert. U.S. Pat. No. 6,074,413 is directed to a disposable thermal back wrap having one or more thermal inserts comprising a plurality of heat cells, wherein heat is applied to specific areas of the user's back for pain relief U.S. Pat. No. 5,605,144 is directed to a heating garment with a pouch for accommodating inserted chemical heating inserts that are air activated.

U.S. Pat. No. 5,484,366 is directed to an aerobic/cross training exercise belt. The belt comprises a straight piece of material having a fastener on each end whereby the ends can be fastened together to form a closed belt. A back lumbar support is connected to the rear body of the belt. The back lumbar support has at least one pocket to mount chemical gel-inserts whereby the user would have a thermal application to the lumbar area while wearing the belt. The gel inserts can be heated or cooled to the desired temperature. U.S. Pat. No. 6,623,419 is directed to a therapeutic back belt and related method of manufacture. The belt includes magnets that are secured to the belt and thermally active gel material. U.S. Pat. No. 5,179,942 is directed to a lumbar support therapeutic heat/cooling/air belt. The support has one pocket in the lower back section that is capable of receiving an insert to create a thermal change or provide air for support purposes.

U.S. Pat. No. 5,925,072 is directed to a disposable elastic thermal insert wherein iron powder based exothermic compositions are segmented into individual portions and integrated into a back belt. In this composition, the thermal conductivity is not optimized since the composition is separated from the skin by a fabric barrier. U.S. Pat. No. 5,918,590 is directed to a specific heat cell unit comprising an iron powder based exothermic composition, wherein a specific exothermic formulation and pocket fill volume are defined.

U.S. Pat. No. 6,146,342 is directed to massage pad having a plurality of randomly actuated pressure inducing elements. The apparatus massages the body by subjecting the body to impacts from reciprocating plungers. The plungers are secured in a flat array within a flexible pad. Each plunger has an associated solenoid device that alternately causes the plunger to project from the pad and to retract within the pad. An electrical circuit includes a power cord and plug assembly, manual controls disposed serially on the cord and plug assembly, and a controller generating operating signals randomly to the solenoids. A heating element is optionally included in the flexible pad, with a suitable controller provided among the controls.

Still other types of devices, as shown in U.S. Pat. No. 7,077,858, include those that use flexible heat exchangers to distribute cooling and heating agents to the skin utilizing electrical heat. U.S. Pat. No. 6,409,748 is directed to a heating pad with removable gel insert that provides rapid initial warming. U.S. Pat. No. 4,846,176 is directed to a thermal bandage having a conformable region that can be placed against the skin to uniformly heat or cool the contacted skin area.

Battery or plug-in powered vibratory devices have also been traditionally employed to deliver pain relief, relax tight muscles, relieve muscles soreness, and joint stiffness. Devices have also been disclosed, as shown in U.S. Pat. No. 7,147,610, that incorporate massaging elements with the heating elements so that they are conveniently available in a single device. Such a device has excess bulk, is non-discreet and requires the use of external power sources (i.e. a junction box) because the heating and massaging elements require electrical power. In addition, although the parts are reusable, electrical elements tend to be non-washable. Published U.S. Patent Application 2004/0082886 is directed to a therapeutic device for relieving pain and stress in the hands and feet. The portable device provides heat and vibratory therapies for the hand or foot.

### SUMMARY OF THE INVENTION

The present invention relates to a device to be worn in close contact with the skin of a human, and includes methods for providing a vibration and heating sensation to human skin, and methods for treating muscle aches and pains in a human, as well as a therapeutic device comprising a thermal insert. The thermal and vibratory inserts, devices, and methods of the present invention are useful in managing muscle and joint pain.

The present invention also relates to the combination of thermal and vibratory components for wearing in a garment in close contact with the skin of a human. The present invention also provides a therapeutic device comprising a combined thermal and vibratory insert and a garment. The present invention also provides methods for treating muscle aches and pains in a human.

The present invention relates to a thermal device for topical application on humans that includes a thermal insert containing a thermal composition; a vibrational means that transmits vibrational energy to and through the thermal insert; and a power source having capacity to power the vibratory element from at least about 10 minutes to a maximum duration of about 12 hours. The vibratory element has a maximum dimension not greater than about 7 centimeters. Additionally, the thermal device is a unitary system having a thickness of not greater than about 10 millimeters. Thermal insert can be an air-activated exothermic powder mixture. The thermal device can further maintains a temperature differential of from about 3 to about 10 degrees Celsius for at least about 8 hours, as between the surface of the thermal insert and the skin. The vibrational means can be, at least in part, powered by an electric-powered motor, which optionally can operate on a direct current. The vibrational means can preferably transmit vibrational energy at a frequency of from about 5 H_{z} to about 1000 H_{z}. The thermal insert can additionally comprises at least one thermally conductive component. Such a thermally conductive component can have a hardness as measured using a durometer of greater than or equal to 40 on an A scale. The surface of the vibratory component can have a hardness as measured by durometer of greater than or equal to 40 on an A scale.

The present invention is directed to a method of treating pain in a human patient by providing a device described above, wherein said device delivers both heat sufficient to raise the skin surface temperature to between about 39.5° C. and about 42° C. for at least about 8 hours, and vibration sufficient to be sensed by the patient for at least about 10 minutes to a maximum of about 12 hours. The vibration can be activated by removing a pull-tab or by a switch and can be turned off by the patient. The device is preferably substantially inaudible to the patient while delivering vibration. For example, the vibration can be delivered with a loudness of less than 60 decibels.

The present invention is directed to a system for providing wearable pain relief therapy to a consumer by providing the consumer with a self contained thermal device described above, together with a reusable wrap having pockets for inserting the self contained thermal device. In one embodiment, the wrap is shaped to fit a particular body part, such as the lower back, knee, wrist, neck, shoulder, elbow, ankle.

The present invention is also directed to a system for providing wearable pain relief therapy to a consumer by providing the consumer with a device as described that has been integrally incorporated into a disposable garment that comprises a non-woven fabric.

The present invention is also directed to a method for treating muscle aches and pains in a human by wearing the device described above for a period of time of about 1 hour to about 16 hours.

### DETAILED DESCRIPTION OF THE INVENTION

The thermal and vibratory device of the present invention will typically be worn in a garment. Suitable garments include belts, back belts, back wraps, sleeves, knee sleeves, elbow sleeves, knee or elbow wraps or supports, neck wraps, neck sleeves, shoulder vest, shoulder support, wrist sleeve, wrist support, ankle sleeve, ankle wrap, foot support, sock, glove, hand support, or other braces and supports typically used to stabilize a joint. Suitable garments also include articles designed to adhere to the skin, such as a patch. The garment can be re-usable, e.g. constructed from washable fabric, such as a nylon-spandex fabric. Alternately, the garment can be disposable, e.g. constructed from non-woven materials. The garment preferably comprises a pocket for holding the thermal and vibratory insert. The pocket is preferably constructed of a breathable and porous fabric, and attached to the garment on the surface that will be worn next to the skin. In one particular embodiment, the pocket shape is contoured similarly to the shape of the thermal insert. In embodiments wherein the garment is designed to adhere to the skin, adhesive can be applied continuously over one surface of the patch-style garment, or adhesive can be applied discontinuously to the edges of the garment. The adhesive can be designed to adhere to the skin, or alternately can be designed to adhere to the interior of the user's clothing. The patch-style garment can be shaped like a sleeve or tube for inserting the thermal insert, or can be a flat piece of fabric with an attached pocket. In one embodiment, the patch-style garment is constructed from a disposable, breathable, non-woven fabric.

The present invention describes a unitary system. As used herein “unitary” defines a device with an integrated thermal insert and a vibratory element.

The thermal insert of the present invention comprises a thermally active component and a thermally conductive component. The thermally active component generates heat or cold for purposes of this invention, preferably including therapeutic purposes. The thermally conductive component improves the efficiency of delivery of said heat or cold, and thereby, improving the experience of the user. In one embodiment, the thermal insert can comprise a thermally active composition, which is a component, material or combination of materials that activates upon the addition of heat or cold, thereby produces the heat or cold; a thermal fill composition, or combinations thereof In one embodiment, the thermal insert comprises an enclosure for said thermally active composition.

The thermal insert can comprise a thermal composition that can be any material suitable for either generating, or holding heat or for maintaining a low (cold) temperature. In one embodiment, the thermal composition emits heat from about 1 to about 10 degrees Celsius above the skin surface temperature of a human. In an alternate embodiment, the thermal composition maintains a temperature from about 1 to about 20 or 50 or up to 100 degrees Celsius lower than the skin surface temperature of a human.

In one particular embodiment, the thermal insert comprises a thermal composition that is a mixture of substances that react exothermically. For example, several commercial hand warmers and therapeutic heat products contain an iron powder based mixture that liberates heat as the iron is oxidized upon exposure to air. These types of systems are described in detail in for example, U.S. Pat. No. 5,918,590. It is known in the art to formulate these mixtures to maintain a temperature of at least about 40 degrees Celsius for at least 4 hours, and up to 24 hours, for example, for at least about 8 hours, e.g. for at least about 10 hours, say for at least about 12 hours, or for at least about 16 hours.

In another embodiment, the thermal insert comprises a thermal fill material that is capable of absorbing microwave energy and retransmitting such energy as heat energy. Suitable microwave energy retaining fill materials include rice, corn, barley, cherry stones, starch-based synthetic pellets, and the like. Such materials typically retain a suitable level of heat for about 20 to about 60 minutes.

In another embodiment, the thermal insert comprises electrically heated or electrically cooled articles, such as a resistive heater, or a thermoelectric based cooling and heating element such as Peltier element.

In certain embodiments, the temperature measured by a thermocouple inserted between the individual's skin and the thermally conductive component of the thermal insert of this invention is 37° C., 38° C., 40° C., 41° C., 45° C., or 50° C.

In another embodiment, the thermal insert comprises a thermal composition that is a liquid that changes phase, such as solidifies or gels at a desired temperature. Upon storage in a freezer, for example, the material solidifies and maintains a temperature of less than about 5 degrees Celsius for about 20 to about 90 minutes. In one such embodiment, the temperature measured by a thermocouple inserted between the individual's skin and the thermally conductive member of the thermal insert of this invention is 5° C., 10° C., 20° C., 25° C., 30° C., or 35° C.

In one embodiment, the thermal insert is a material or combination of materials that are solid from about −20° C. to 20° C., or at about 0° C. In one embodiment, the thermal insert is substantially free of a material that is combustible, flammable, or volatile. As used herein, “substantially free” is defined as less than 1 percent by weight of the thermal insert. Combustible materials include but are not limited to fuels such as alcohols, such as ethanol, methanol and butanol; or fuels, such as lighter fluids, kerosene, lantern oils, and mixtures thereof

In one embodiment, the thermal insert comprises an enclosure. The optional enclosure for the thermal insert can be any material that contains the thermal composition within the thermal insert. In one embodiment, the enclosure is a pouch constructed of breathable non-woven fabric. In another embodiment, the enclosure is a water-tight polymer film pouch for holding a freezable liquid. In another embodiment, the enclosure is constructed from woven textile fabric. In certain embodiments, the enclosure is a pouch having one surface formed from a relatively non-conductive fabric, and a second surface comprising the thermally conductive component.

The thermally conductive component has a thermal conductivity of at least about 10 W/mK, such as at least about 100 W/mK, say from about 150 W/mK to about 250 W/mK. For the sake of comparison, the thermal conductivities for some representative materials are shown below:

| | |
|---|---|
| Polypropylene: | 0.12 W/mK |
| Stainless steel: | 21 W/mK |
| Aluminum: | 221 W/mK |

Suitable materials for forming the thermally conductive component include metals, such as aluminum, copper, silver, steel, and metal alloys of aluminum, copper, silver, steel, and combinations thereof; non-metallic thermally conductive materials, such as carbon-based materials, including graphite, glassy carbon, thermally conductive plastics, polymers, rubber, or such as conductive textiles, composites, ceramics, and mixtures thereof. Optionally, these thermally conductive components can contain wires or fibers comprising the metals described above in order to make them more thermally conductive. Preferably, the thermally conductive component is non-reactive with the thermal composition, or with air and moisture.

In embodiments in which the thermal insert comprises a material that is activated by microwave, the thermally conductive component must be designed accordingly. For example, in one version of this embodiment, the thermally conductive component comprises a non-metallic substance such as ceramic. In another version of this embodiment, the thermally conductive component comprises a plastic portion that has a metallic surface underneath that does not expose metal to the microwave. In yet another version of this embodiment, the thermally conductive component is packaged separately from the thermally active component, along with means (such as an adhesive) for attaching the thermally conductive component to the thermally active component after microwave heating.

In certain preferred embodiments, the thermally conductive component has a portion of its surface that extends from and above the plane of the thermally active component. In certain such embodiments, the raised portions have a rounded shape. As used herein, rounded shape is defined as elliptical, semi-elliptical, semi-circular, or circular. In certain such embodiments, the raised portions of the thermally conductive component are raised by from about 2 millimeters to about 3 centimeters above the surface of the thermally active component or enclosure for such thermally active component. The raised portions of the thermally conductive component can advantageously provide a massaging sensation when held against the skin. For example, when the thermal insert of the present invention is worn in a back belt, with the raised portions of the thermally conductive component in close contact with the skin, the raised portions can give the sensation of fingers, massaging the skin as the wearer moves. In one particular embodiment, all or a portion of the thermally conductive component can be configured to rotate around a supporting element, or within a socket. In this embodiment, the thermally conductive massaging element can be shaped as a cylinder, sphere, octahedron, dodecahedron, or any suitable rotatable shape.

In a broader embodiment, the thermally conductive component can be of a various shapes, including round, semi-spherical, elongated, ellipsoidal, cylindrical, star shaped, mushroom shaped, or similar shapes. According to an embodiment of the present invention, the shapes of the thermally conductive component at the interfaces to the individual's body can be flat or non-flat, including but not limited to semi-spherical, pyramidal, conical, concave, convex, bumped, or contain an array of smaller shapes, e.g. semi-spherical protrusions.

In certain embodiments, the thermally conductive component can form a single, continuous layer on the surface of the thermally active component. For example, the thermally conductive component can be a single piece of foil having deep drawn protrusions in its surface. In certain other embodiments, the thermally conductive component can be discontinuously arranged upon a surface of the thermally active component. For example, the thermally conductive component can be a single piece of foil having cut-outs to enhance aesthetics or breathability of the thermal insert, or the thermally conductive component can comprise a plurality of individual metallic parts, individually adhered to the surface of the enclosure for the thermal fill composition. In embodiments where the thermally conductive component is a piece of foil, the thickness of the foil can be from about 0.006 mm to about 0.5 mm, or about 0.01 mm to about 0.2 mm. The foil can be present on a single surface of the thermal insert, on two or more surfaces or surrounding the entire thermal insert.

In certain embodiments, the thermally conductive component can itself form a portion of the enclosure for the thermal fill composition. For example, the thermal fill composition can be a powder enclosed in a pouch-type structure, one surface of which comprises a porous non-woven fabric, and another surface of which comprises a metallic thermally conductive material, or the thermal fill composition can be a freezable liquid or gel enclosed in a pouch-type structure, one surface of which comprises a polymeric water-tight film, and another surface of which comprises a metallic film.

In another such embodiment, the thermal insert can be configured so that a portion of the thermally conductive component is in contact with the thermal fill material and the interior of the enclosure, while another portion of the thermally conductive component protrudes through openings in the enclosure to form an exterior surface.

The thermally conductive component can be rigid, or soft and compressible. In embodiments employing the thermally conductive component to deliver a massaging sensation, the massaging elements of the thermally conductive component are preferably rigid enough to maintain their shape when pressed against the skin. The raised portions of the thermally conductive component can be solid, hollow, or filled with conductive or non-conductive material. In one embodiment, the interior surface of the raised portions of the thermally conductive component are in contact with the thermal fill material. In one embodiment, the thermally conductive component is filled with metal pellets.

Another aspect of the present invention relates to methods for treating or managing pain, particularly muscle or joint pain, in humans. While heat, and massage have long been recognized as effective modalities for managing pain, the thermal insert of the present invention, in the embodiment wherein the thermally conductive component has at least a portion of its surface raised above the plane of the thermally active component, provides a means for delivering heat, along with a massaging sensation to the user. Compared to other methods of providing heat and massage, the method of the present invention is advantageously portable, wearable, and long lasting, with minimal effort required on the part of the user.

Yet another benefit of the massaging action of the thermal insert of the present invention is the sensory cue to remind the user the product is working. Sensory cues can improve patient compliance with a treatment regimen. One such regimen includes the wearing of the thermal insert of the present invention in close contact with the skin (either via a garment or patch) for from about 1 to about 16 hours. For example, a user can wear the thermal insert for from about 4 hours to about 8 hours, or from about 8 hours to about 12 hours, or from about 8 hours to about 16 hours, providing heat to the affected muscles or joints while simultaneously engaging in work or leisure activities.

In the therapeutic use of the thermal inserts of the present invention, the thermally conductive components are in contact with the body of the user, either directly contacting the skin, or contacting the body through clothing or garments worn by the user. Simultaneously the thermally conductive components are in contact with the thermal insert. The thermally conductive component serves to effectively transfer or re-distribute heat or cold from the thermal inserts to the individual's body. In addition, thermally conductive component create a non-uniform thermal sensations on the body or on the skin in case of direct application to skin, whereby body or skin areas in immediate contact with the thermally conductive component experience much stronger sensations of heat or cold relative to the adjacent areas.

In one embodiment, the thermally conductive member is substantially free of activated carbon, e.g. less than 0.1% by weight of the fill of the thermally conductive component.

In one embodiment (not shown), the interior cavities created by raised portions of the thermally conductive component are filled with substances that are capable of retaining heat for extended periods of time, such as thermal beads, encapsulated water, wax, phase changing materials, ceramics, sand, grains, rice, wheat, corn, etc. Even after the chemical formulation inside the thermally active component stops delivering or generating heat, the substances that are capable of retaining heat for extended periods of time filled inside the thermally conductive component can continue releasing or absorbing heat for extended periods of time. Additionally, in case of accidental overheating of the chemical formulation inside the thermally active component, said substances are capable of absorbing the excess heat thus providing protection form overheating.

Advantageously and beneficially, the space around the raised portions of the thermally conductive component is available for removal and evaporation of sweat and provides for areas of the body or skin not in contact or not covered by any implement. Additionally, thermal contrast delivered to the body can be much higher whereby thermally conductive component transferring heat and transferring cold can be immediately adjacent to each. This is also achieved without significant losses of thermal energy due to heat transfer.

In one embodiment, the thermal insert is a thermal pack.

The number of the thermally conductive component(s) per single thermal pack can vary from one to several. In one embodiment, from 6 to 30 or more thermally conductive components are installed on the thermal pack. In one embodiment, the dimensions of the thermally conductive components have a width from about 5 millimeters to about 50 millimeters, e.g. from about 7 millimeters to about 20 millimeters. In one embodiment, the dimensions of the thermally conductive components have a height from about 5 millimeters to about 50 millimeters, e.g. from about 7 millimeters to about 20 millimeters.

In embodiments wherein the shape of the thermally conductive components are semi-spherical, the diameter, which is equal to the width of the component, is from about 5 millimeters to about 50 millimeters, e.g. from about 10 millimeters to about 30 millimeters. In this embodiment, the radius of the semi-spherical component, which is equal to the height, is from about 2.5 millimeters to about 25 millimeters, e.g. from about 5 millimeters to about 20 millimeters.

In certain embodiments, the thermally conductive components can be defined by the volume of the internal space of the component. In certain embodiments, the internal volume of a thermally conductive component can be from about 0.01 milliliters to about 50.00 milliliters, e.g. from about 0.03 milliliters to about 33.00 milliliters, e.g. from about 0. 10 milliliters to about 2.00 milliliters.

In one embodiment wherein more than one thermally conductive components is present in the device, all thermally conductive components have the same height, while in another embodiment, some thermally conductive components are higher and some are lower, for example a first portion of the thermally conductive components are about 5 millimeters to about 10 millimeters high, while a second portion are about 10 millimeters to about 15 millimeters high, while an optional third portion are about 15 millimeters to about 20 millimeters high.

In one embodiment, the surface of the thermally conductive component is harder than the surface of the skin as measured by a Shore Durometer on the Type A scale. The term durometer as used herein refers to the measurement as well as the instrument itself In this embodiment, the durometer of the surface of the thermally conductive component is greater than or equal to 40 on the Type A scale, or greater than or equal to 80 on the Type A scale.

The thermal device can be of any shape and size suitable for wearing next to the skin of a human, and can be produced commercially in any shape and size that can be die cut. For example, the thermal insert can be round, triangular, square rectangular, pentagonal, hexagonal, etc. In one embodiment, at least one dimension of the thermal devices is from about 1 inch to about 30 inches. In one particular embodiment, the thermal insert of the device has a triangular shape with a width from about 2 to about 6 inches, and overall length from about 2 to about 12 inches. In one particular embodiment, the thermal insert of the device has a hexagonal shape.

In certain embodiments, the thermal device can be substantially flat with the thickness of the device ranging from about 2 millimeters to about 30 millimeters, and the other dimensions of the insert ranging from about 24 millimeters to about 720 millimeters.

The vibratory massaging element can be any vibrating mechanism, including an electric motor with offset weight located on the rotor, stop-and-go electric motor, linear motors operated in a back and forth fashion, piezo-electric vibrator, electric solenoid vibrator, air or water pump based vibrator, inflatable flexible pulsating bulb, or other vibrator type known in the art.

The term vibration as used herein refers to mechanical oscillations about or from an equilibrium point or starting point. The oscillations can be periodic such as the motion of a pendulum, random, or pulsatile with variable dwell and pulse periods and variable amplitudes.

Vibration, as used herein, is intended to be desirable. For example, the motion of a tuning fork, the reed in a wood wind instrument, or the cone of a loudspeaker is desirable vibration, necessary for the correct functioning of the various devices. In certain types of devices, vibration is undesirable, can waste energy and can create unwanted sound noise. For example, the vibrational motions of electric motors, and engines or any mechanical device in operation are typically unwanted. Such vibrations can be caused by imbalances in the rotating parts, uneven friction, the meshing of gear teeth, etc. Careful design in these types of devices usually minimizes unwanted vibrations.

The science of sound and vibration are closely related. Sound and pressure waves are generated by vibrating structures (e.g. vocal cords) and pressure waves can generate vibration of structures (e.g. ear drum). Hence, a reduction in noise emanating from a device is most often achieved by reducing the associated vibration. In the case of this invention, it is desirable to increase vibration without increasing audible undesirable sounds.

The traditional definitions of vibration are categorized as either Free Vibration, or Forced Vibration. “Free vibration”, as used herein, occurs when a mechanical system is set off with an initial input and then allowed to vibrate freely. Examples of this type of vibration are pulling a child back on a swing and then letting go or hitting a tuning fork and letting it ring. The mechanical system will then vibrate at one or more of its natural frequencies and damp down to zero. Forced vibration as used herein is defined as an alternating force or motion that is applied to a mechanical system. Examples of this type of vibration include shaking washing machine due to an imbalance, transportation vibration (caused by truck engine, springs, road, etc), or the vibration of a building during an earthquake. In forced vibration, the frequency of the vibration is the frequency of the force or motion applied, but the magnitude of the vibration is strongly dependent on the mechanical system itself.

In one embodiment of the present invention, the vibratory element can function as a free vibration system. In one embodiment, the vibratory element can function as a forced vibration system. In another embodiment, the vibratory element can function as a combination of forced and free vibration. In one embodiment, the combination of a forced and free system incorporates a resonating element such as a tuning fork, tuning rod, tuning plate, spinning eccentric mass on a shaft, resonating cavity etc. that is energized with forced vibration or rotational input and allowed to vibrate or rotate freely. After the vibration has stopped, it is energized again and allowed to decay. In this way energy is conserved in the conversion from kinetic to potential energy, thus maximizing force and conserving input energy which is desirable in portable battery powered embodiments.

There are two means whereby the sensation of a vibrational massage are achieved. “Vibrational massage”, as used herein, is delivered in one embodiment using direct-acting systems and in a second embodiment using coupled systems.

An embodiment of a direct acting system is comprised of a motive force such as a linear motor, solenoid, or rotary motor with eccentric and connecting rod attached to a linear bearing, all of which is oriented with their axis that is roughly perpendicular to the body. The actuator is constrained at one end portion, whereas the other end portion is in contact with and presses against the skin or body. The constrained end can be held by mechanical means such as a band, belt, adhesive attachment, hand pressure, or an external surface such as a chair back or seat pad. A periodic or random input is given to the actuator (various wave forms are possible such as sinusoidal, square wave, triangular wave and random amplitude and period wave forms) resulting in an in and out (or extend—retract) motion.

The coupled vibratory systems are the second classification of embodiments described herein. The function can be easily visualized using the classic mass, spring, damper model cited in physics and vibration analysis texts. In this type of system, the vibrational element provides forced excitation and the human body becomes part of the spring mass damper system. In general, the body is equivalent to the spring and viscous damper of the model as well as an additional mass. In this embodiment, the human body or skin surface is the damper. The damper sees a force that is proportional to the velocity of the moving mass of the vibrational element. The damping is called viscous because it models the effects of an object in a fluid. In one embodiment, the skin sees a force equal to the reaction force of mass moving during the vibration sequence.

The massage sensation is achieved through the constraint of the moving mass mechanism against the body whereby the damping effect results in a force acting upon the skin. In a preferred embodiment, the thermal insert is a coupling element. The coupling element herein refers to the element generally transmitting at least some of the movement or vibration to the body. That is the coupling element at least in part interfacing between the vibratory element and the body. The coupling element is in direct contract with the body. The coupling element is not generating the vibration itself, but it is vibrating due to the connection to the vibrating element.

In certain preferred embodiments, the thermally conductive protrusions provide a pathway for vibrational waves to propagate from the device to the skin creating a vibrating sensation.

In a preferred embodiment of the coupled vibratory system, a rotary motor with an eccentric mass attached to a shaft is employed. The rotation of the eccentric mass about the axis of the motor creates the vibrator effect, the speed of rotation establishing the frequency of vibration. The motor and mass preferably are contained in an enclosure to shield the rotating members from disturbance. The degree of force can be tuned by adjusting or selecting the following parameters: speed (frequency of oscillation) amount of eccentric mass, degree of offset of mass from centerline and amount of moving mass. The orientation of the motor axis can be either perpendicular or parallel to the skin surface. The mass, the geometry and the frequency of operation of the system can be optimized to take advantage of resonant effects which will beneficially amplify the input frequency to achieve higher amplitudes and massaging force. When the rotational speed of the motor matches the resonant frequency of the motor, mass, and housing structure the resonating effect is produced.

An example of a vibratory system includes commercially available vibratory elements, such as low voltage direct current (dc) motors with eccentric weights that are designed for commercial products such as mobile phones, electric tooth brushes, toy vehicles, and massagers. They are commercially available from suppliers such as Panasonic, Matsushita, Shicoh and Jin long Machinery. The vibratory element is co-located with the thermal insert, and preferably has a small size, in order to be worn discreetly on the body of the user. Suitable dimensions for the vibratory element are not more than about 7 cm, e.g. not more than about 2 cm, in length, and not more than about 1 cm, e.g. not more than about 0.5 cm in depth. In one embodiment, the vibratory component is incorporated into the thermally conductive component. In one embodiment, the surface of the vibratory component is harder than the surface of the skin as measured by a Durometer on the Type A scale. The term durometer as used herein refers to the measurement as well as the instrument itself. In this embodiment, the durometer of the surface of the vibratory component is greater than or equal to 40 on the Type A scale, or greater than or equal to 80 on the Type A scale.

In one embodiment, the power source is contained within the thermal device, and has capacity to power the vibratory element for at least about 20 minutes, e.g. at least about 30 minutes, of operation. Preferably the power source is small in size, in order to be contained conveniently and worn discreetly on the body of the user. In certain embodiments, the power source can be a single-use battery, such as either button cell shaped or cylindrical cell shaped or flat-pouch encapsulated battery based on any chemical composition known in the art, including manganese-zinc, metal hydride, lithium, lithium ion, lithium polymer, and other known battery chemistries. Voltage of the battery can range from 1 volts to 3 volts to 12 volts depending on the motor employed. The battery capacity is selected to provide for sufficient time of operation of the vibrating element as described above. Suitable power sources can include primary batteries and secondary (rechargeable) batteries, fuel cells, hydrocarbon fuel cells, printed batteries, and plug-in power sources. In one embodiment, the power source for the vibratory component must have adequate energy density for the vibratory component to vibrate for up to 8 hours, e.g. up to 10 hours, e.g. up to 12 hours.

In one embodiment, the vibratory element can be activated by completing an electrical circuit with the power source. In certain embodiments, this can be achieved by means of a pull-out tab separating two contacts thus closing the electric circuit and activating the vibrating element, as known in the art. This prevents electrical communication during shipping and storage of the device, but allows electrical communication when removed by the user. In certain other embodiments, the activation of the vibratory element can be achieved by a magnet-based actuator, such as magnet-sensitive proximity switch, by an electric switch, or similar electric circuit activation element. In one embodiment, the vibratory element or thermal device can also optionally incorporate a time-controller, which will switch the vibration off after a pre-set period of time, for example after 20 minutes or 30 minutes or after 1 hour. In another embodiment, the vibratory element may have extensions connected from a central vibratory source that delivers the vibratory sensation over a portion of the surface of the device or over the entire surface of the device, as a means for increasing the surface area of the vibratory sensation over the skin. These extensions may be present as fingers or plates. A vibratory resonance may be incorporated between the source and the extensions to increase the force of the total vibration or to modulate the frequency of the vibration.

In certain embodiments, the vibratory element is operational for a relatively short time period, compared to the life of the thermal insert. In one particular embodiment, the vibratory element delivers a vibration for the initial 20 minutes to 30 minutes of operation, then stops. Short periods of vibration can enhance the perceived benefit from the heat, especially helping to loosen stiff muscles before the thermal insert reaches its maximum temperature. In one embodiment the ratio of time for the vibratory element to the thermal insert is 1:50 or 1:25 or 1:10 or 1:5.

In certain embodiments it is desirable to minimize the noise level from the vibratory element. The noise level depends upon the vibrational frequency, the damping provided by the skin of the user or the enclosure; the mass of the vibrational element and eccentric distance traveled by the eccentric mass on the rotary motor. In certain embodiments, the device is substantially inaudible to the patient while delivering vibration. In these embodiments, the frequency of vibration is preferably below about 150 Hz, for example below about 20 Hz., however higher frequencies up to about 10,000 Hz can be utilized as the noise is somewhat dampened by the vibrating element being enclosed and being in contact with sound-dampening soft tissue and heating element.

Preferred amplitude and frequency ranges of vibration are selected to provide optional patient's relief and to utilize low cost, miniature vibrators. Preferably, the vibration can be sensed at the skin surface, but is substantially inaudible to the patient. In one embodiment, the frequency is from about 1 Hz to about 10,00 Hz, another embodiment from about 5 Hz to about 1,000 Hz, and in yet another embodiment, from about 1 Hz to about 100 Hz. The amplitude of vibratory stimulation as measured on the surface of the device, ranges from 0.1 mm peak-to-peak to 1 mm peak-to-peak and up to 15 mm peak-to-peak. The human auditory system is sensitive to frequencies from about 20 Hz to a maximum of around 20,000 Hz, although the hearing range decreases with age. Within this range, the human ear is most sensitive between 1,000 Hz and 5,000 Hz, largely due to the resonance of the ear canal and the transfer function of the ossicles in the middle ear. One desired function of the vibratory element is to create no perceptibly audible disturbance to the user.

Equal-loudness contours were first measured by Fletcher and Munson using headphones (1933). In their study, listeners were presented with pure tones at various frequencies and over 10 dB increments in stimulus intensity. For each frequency and intensity, the listener was also presented with a reference tone at 1000 Hz. The reference tone was adjusted until it was perceived to be of the same loudness as the test tone. Loudness, being a psychological quantity, is difficult to measure, so Fletcher and Munson averaged their results over many test subjects to derive reasonable averages. Based on these studies human hearing sensitivity was found to drop off dramatically below 100 Hz.

In a preferred embodiment, the loudness of the noise from the vibratory element is preferably below 60 dB at a distance of 1 foot which is equivalent to human conversation, such as below 50 db at one foot, or below 20 db at 1 foot.

The voltage of the power supply is selected to be safe and to provide sufficient power to the vibratory element. Typically the voltage is from 1.5 V to 3 V, and in some embodiments up to 12 V DC.

I/We claim:

1 A thermal device for topical application on humans that comprises:
a) a thermal insert containing a thermal composition;
b) a vibrational means that transmits vibrational energy to and through the thermal insert; and
c) a power source having capacity to power the vibratory element from at least about 10 minutes to a maximum duration of about 12 hours;
wherein the vibratory element has a maximum dimension not greater than about 7 centimeters; and the thermal device is a unitary system having a thickness of not greater than about 10 millimeters.

2. The thermal device of claim 1, wherein the thermal insert comprises an air-activated exothermic powder mixture.

3. The thermal device of claim 1, which maintains a temperature differential from about 3 to about 10 degrees Celsius for at least about 8 hours, as between the surface of the thermal insert and the skin

4. A device according to claim 1 wherein the vibrational means comprises an electric-powered motor.

5. A device according to claim 4 wherein the electric power mower operates as a direct current.

6. A device according to claim 1 wherein the vibrational means transmits vibrational energy at a frequency of from about 5 H_{z} to about 1000 H_{z}.

7. A device according to claim 1 wherein the thermal insert additionally comprises at least one thermally conductive component.

8. A device according to claim 7 wherein the thermally conductive component has a durometer of greater than or equal to 40 on a Type A scale.

9. A device according to claim 1 wherein the surface of the vibratory component has a durometer of greater than or equal to 40 on a Type A scale.

10. A method of treating pain in a human patient, comprising providing the patient a device according to claim 6, wherein said device delivers both heat sufficient to raise the skin surface temperature to between about 39.5° C. and about 42° C. for at least about 8 hours, and vibration sufficient to be sensed by the patient for at least about 10 minutes to a maximum of about 12 hours.

11. The method of claim 10, wherein the vibration is activated by removing a pull-tab.

12. The method of claim 10 wherein the vibration is activated by a switch and can be turned off by the patient.

13. The method of claim 10 wherein the device is substantially inaudible to the patient while delivering vibration.

14. The method of claim 10 wherein the vibration is delivered with a loudness of less than 60 decibels.

15. A system for providing wearable pain relief therapy to a consumer, comprising providing the consumer with the self contained thermal device of claim 1, together with a reusable wrap having pockets for inserting the self contained thermal device.

16. The system of claim 15 wherein said wrap is shaped to fit a particular body part, selected from the group consisting of lower back, knee, wrist, neck, shoulder, elbow, ankle.

17. A system for providing wearable pain relief therapy to a consumer, comprising providing the consumer with the device of claim 1, which has been integrally incorporated into a disposable garment, comprising a non-woven fabric.

18. A method for treating muscle aches and pains in a human comprising wearing the device of claim 1 for a time period of about 1 hour to about 16 hours.
